(19) United States
(12) Patent Application Publication
(10) Pub. No.: US 2007/0088346 A1
(43) Pub. Date: Apr. 19, 2007

(54) Method and apparatus for varicose vein treatment using acoustic hemostasis

(76) Inventors: Michael S. Mirizzi, San Jose, CA (US); Arthur W. Zikorus, San Jose, CA (US); Brady D. Esch, San Jose, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/581,596

(22) Filed: Oct. 16, 2006

Related U.S. Application Data
(60) Provisional application No. 60/726,999, filed on Oct. 14, 2005.

(51) Int. Cl. A61B 18/04

(52) U.S. Cl. 606/27

(57) ABSTRACT

A method of treating a perforator vein comprises applying ultrasound to the perforator vein and occluding the perforator vein with the ultrasound. An apparatus for treating blood vessels comprises an ultrasound emitter, wherein the ultrasound emitter is configured to emit ultrasound at multiple therapeutic ultrasound frequencies during a treatment cycle. The apparatus further comprises an acoustic coupler in sonic communication with the emitter, wherein the acoustic coupler has an acoustic coupling surface configured to contact a patient and facilitate delivery of ultrasound to the patient and wherein the acoustic coupler provides a conduction path for ultrasound from the emitter to the acoustic coupling surface. The apparatus further comprises an acoustic coupler containing a displaceable acoustic coupling material. The apparatus further comprises an acoustic coupler configured to vary the length of the conduction path in accordance with variation in the thickness of the acoustic coupling material disposed between the emitter and the acoustic coupling surface.

### CROSS-REFERENCE TO RELATED APPLICATIONS

This application claims the benefit under 35 U.S.C. § 119(e) of U.S. Provisional Application No. 60/726,999, filed Oct. 14, 2005, titled METHOD AND APPARATUS FOR VARICOSE VEIN TREATMENT USING ACOUSTIC HAEMOSTASIS. The entire contents of the above-listed provisional application are hereby incorporated by reference herein and made part of this specification.

### BACKGROUND

1. Field of the Invention

Certain embodiments disclosed herein relate to a method and apparatus for applying energy to constrict or shrink a hollow anatomical structure such as a vein, and more particularly, a method and apparatus to apply high intensity focused ultrasound to treat a hollow anatomical structure.

2. Description of the Related Art

The human venous system of the lower limb consists essentially of the superficial venous system and the deep venous system with perforating veins connecting the two systems. The superficial system includes the long or great saphenous vein and the short saphenous vein. The deep venous system includes the anterior and posterior tibial veins which unite to form the popliteal vein, which in turn becomes the femoral vein when joined by the short saphenous vein.

The venous systems contain numerous one-way valves for directing blood flow back to the heart. Venous valves are usually bicuspid valves, with each cusp forming a sack or reservoir for blood which, under pressure, forces the free surfaces of the cusps together to prevent retrograde flow of the blood and allow antegrade flow to the heart. When an incompetent valve is in the flow path of retrograde flow toward the foot, the valve is unable to close because the cusps do not form a proper seal and retrograde flow of blood cannot be stopped.

Incompetence in the venous system can result from vein dilation, which causes the veins to swell with additional blood. Separation of the cusps of the venous valve at the commissure may occur as a result. The leaflets are stretched by the dilation of the vein and concomitant increase in the vein diameter which the leaflets traverse. Stretching of the leaflets of the venous valve results in redundancy which allows the leaflets to fold on themselves and leave the valve open. This is called prolapse, which can allow reflux of blood in the vein. Eventually the venous valve fails, thereby increasing the strain and pressure on the lower venous sections and overlying tissues. Two venous diseases which often involve vein dilation are varicose veins and chronic venous insufficiency.

### SUMMARY

In certain embodiments, a method treats a perforator vein. The method comprises applying ultrasound to the perforator vein. The method further comprises occluding the perforator vein with ultrasound. In certain embodiments, ultrasound is high intensity focused ultrasound (HIFU). In certain embodiments, occluding the perforator vein comprises occluding the perforator vein at a location below or near the deep fascia of a leg. In another embodiment, occluding the perforator vein comprises occluding the perforator vein at a location below the deep fascia of a leg. In certain embodiments, applying ultrasound to the perforator vein comprises generating heat. In another embodiment, the method further comprises causing tissue necrosis, vein wall collagen contraction, vein diameter reduction and fibrotic occlusion, via generated heat. In certain embodiments, applying ultrasound to the perforator vein comprises disrupting the endothelium of the vein with the ultrasound.

In certain embodiments, the method of treating a perforator vein further comprises applying subfascial, perifascial or subcutaneous tumescent fluid near the perforator vein. In another embodiment, the method further comprises causing compression of the inner walls of the perforator vein toward each other with the tumescent fluid. In certain embodiments, applying ultrasound to the perforator vein comprises propagating the ultrasound through the tumescent fluid. In another embodiment, applying ultrasound to the perforator vein comprises initiating cavitation in or near the perforator vein. In certain embodiments, the method further comprises limiting the depth of ultrasound beam penetration with cavitation.

In certain embodiments, applying ultrasound comprises applying ultrasound at multiple frequencies. In another embodiment, ultrasound is applied via an ultrasound probe. In certain embodiments, the method of applying ultrasound further comprises displacing the ultrasound probe along the direction of propagation of the ultrasound while applying the ultrasound. In another embodiment, the method further comprises manually compressing tissue near the perforator vein with the ultrasound probe. In certain embodiments, manually compressing tissue comprises adjusting the location of a focal point of the ultrasound. In another embodiment, applying ultrasound further comprises applying low-power ultrasound during a low-power application phase and high-power ultrasound during a high-power application phase.

In certain embodiments, a method facilitates perforator vein treatment with an ultrasound-generating vein treatment apparatus. The method comprises configuring the vein treatment apparatus to apply ultrasound to one or more perforator veins. The method further comprises configuring the vein treatment apparatus to occlude one or more perforator veins with the ultrasound. In certain embodiments, the method further comprises configuring the ultrasound-generating vein treatment apparatus to function in both a diagnostic and therapeutic mode. In another embodiment, the method further comprises equipping the ultrasound-generating vein treatment apparatus with a standoff to deliver the ultrasound. In certain embodiments, the method further comprises equipping the ultrasound-generating vein treatment apparatus with a flow path in communication with the standoff to facilitate adjusting a standoff distance via material flow to or from the standoff.

In certain embodiments, a method treats a vein. The method comprises applying tumescent fluid to tissue near the vein. The method further comprises applying ultrasound to the vein. The method further comprises occluding the vein with the ultrasound. In certain embodiments, the ultrasound is high intensity focused ultrasound. In another embodiment, occluding the vein comprises occluding the vein at a location below or near the deep fascia of a leg. In certain embodiments, occluding the vein comprises occluding the vein at a location below the deep fascia of a leg. In another embodiment, applying ultrasound to the vein comprises disrupting the endothelium of the vein with the ultrasound. In certain embodiments, applying ultrasound comprises generating heat. In another embodiment, the method further comprises causing tissue necrosis, vein wall collagen contraction, vein diameter reduction and fibrotic occlusion, via the generated heat.

In certain embodiments, applying the tumescent fluid further comprises applying subfascial, perifascial or subcutaneous tumescent fluid near the vein. In another embodiment, the method further comprises causing compression of the inner walls of the vein toward each other with the tumescent fluid. In certain embodiments, applying ultrasound to the vein comprises propagating the ultrasound through the tumescent fluid. In another embodiment, applying ultrasound to the vein comprises initiating cavitation in or near the vein. In certain embodiments, the method further comprises limiting the depth of ultrasound beam penetration with cavitation. In another embodiment, applying ultrasound comprises applying ultrasound at multiple frequencies. In certain embodiments, the vein is a perforator vein. In another embodiment, applying ultrasound comprises applying low-power ultrasound during a low-power application phase and high-power ultrasound during a high-power application phase.

In certain embodiments, an apparatus treats blood vessels. The apparatus comprises an ultrasound emitter. The ultrasound emitter is configured to emit ultrasound at multiple therapeutic ultrasound frequencies during a treatment cycle. The apparatus further comprises an acoustic coupler in sonic communication with the emitter. The acoustic coupler has an acoustic coupling surface configured to contact a patient and facilitate delivery of ultrasound to the patient. The acoustic coupler provides a conduction path for ultrasound from the emitter to the acoustic coupling surface. The acoustic coupler contains a displaceable acoustic coupling material. The acoustic coupler is configured to vary the length of the conduction path in accordance with variation in the thickness of the acoustic coupling material disposed between the emitter and the acoustic coupling surface.

In certain embodiments, the acoustic coupling material is flowable and the acoustic coupler comprises a flow path configured to allow the acoustic coupling material to flow in or out of the acoustic coupler, to facilitate varying the length of the conduction path. In another embodiment, the flow path extends from the acoustic coupler to a coupling material reservoir. In certain embodiments, the acoustic coupler comprises a compartment containing a fluid or mixture of fluid. In another embodiment, the acoustic coupling material comprises a gel. In certain embodiments, the acoustic coupling material comprises a degassed liquid. In another embodiment, the acoustic coupler is a lens. In certain embodiments, the acoustic coupler comprises a standoff.

In certain embodiments, a method of treating a vein comprises emitting multiple ultrasound frequencies with an ultrasound probe. The method further comprises applying the multiple ultrasound frequencies to a treatment region that includes a portion of the vein. The method further comprises adjusting the distance between the ultrasound probe and the treatment region while applying the multiple ultrasound frequencies. The method further comprises occluding the vein with the multiple ultrasound frequencies.

In another embodiment, emitting multiple ultrasound frequencies comprises creating a pattern of multiple focal areas in the treatment region. In certain embodiments, adjusting the distance comprises moving the pattern of focal areas within the treatment region. In another embodiment, moving the pattern of focal areas comprises moving the pattern along a direction of propagation of the ultrasound. In certain embodiments, the vein is a perforator vein. In another embodiment, the method further comprises applying subfascial, perifascial or subcutaneous tumescent fluid near the vein.

Certain objects and advantages of the disclosed invention(s) are described herein. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

The embodiments summarized above are intended to be within the scope of the invention(s) herein disclosed. However, despite the foregoing discussion of certain embodiments, only the appended claims (and not the present summary) are intended to define the invention(s). The summarized embodiments, and other embodiments of the present invention, will become readily apparent to those skilled in the art from the following detailed description of the preferred embodiments having reference to the attached figures, the invention(s) not being limited to any particular embodiment(s) disclosed.

### BRIEF DESCRIPTION OF THE DRAWINGS

FIG. 1 is a view of one embodiment of an ultrasound probe used to treat body tissue.

FIG. 2 is a view of one embodiment of an acoustic coupler applied between the ultrasound probe and the skin.

FIG. 3 is a view of one embodiment of an acoustic coupler comprising an ultrasound standoff.

### DETAILED DESCRIPTION OF THE PREFERRED EMBODIMENTS

New blood vessel treatment systems and methods have been invented. New treatment systems and methods disclosed herein include noninvasive apparatus and methods for occluding blood vessels, particularly veins. Non-invasive surgical procedures have been developed to treat varicose veins. One such procedure utilizes ultrasound to create an occlusion in the blood vessels in order to prevent blood flow through the vessel. Specifically, ultrasound treats perforator veins through the application of ultrasound to the perforator vein and the occlusion of the perforator vein with ultrasound. Additionally, perforator vein treatment is facilitated by use of an ultrasound-generating vein treatment apparatus that is configured to apply ultrasound to the perforator vein and to occlude the perforator vein with the ultrasound.

To treat varicose veins, an ultrasound probe, which is capable of both diagnostic and therapeutic modalities in some embodiments, is located over the vessel intended for treatment and/or used completely extracorporeally so as to obviate the need for making any surgical incisions to expose target tissues or vessels and/or to introduce therapeutic devices into the body. A probe is placed on or near the surface of the skin. By positioning the probe, the ultrasound travels below the skin and results in occlusion of the vein at a location below or near the deep fascia of the skin.

Incompetent perforators are identified and treated, preferably with a single handheld probe with both diagnostic and therapeutic capabilities. Although preferred, extracorporeal use is not required and devices for use on exposed tissues or vessels and/or for introduction into the body can be employed. In some embodiments, the ultrasound probe is displaced along the direction of propagation of the ultrasound while applying the ultrasound. Although the technique is applicable for perforating veins in the lower limb, the technique is easily capable of effecting hemostasis in other blood vessels including veins and arteries as well as other hollow anatomical structures. Alternative clinical applications include using the herein-described methods and apparatus for performing tubal ligation, and treating esophageal varicies, varicoceles, ovarian vein, or fallopian tubes. In some embodiments, the methods are applicable to all hollow anatomical structures.

In this application, the ultrasound used for treatment of veins is of a higher intensity and is more focused. Advances in ultrasound imaging coupled with high intensity focused ultrasound (HIFU) have demonstrated that HIFU is an effective non-invasive modality for effecting hemostasis. HIFU treats veins of the lower limb by providing a non-invasive way to treat patients with varying degrees of venous insufficiency. Specifically, in accordance with methods and apparatus disclosed herein, HIFU can be used to treat perforator veins through the application of ultrasound to the perforator vein and the occlusion of the perforator vein with ultrasound.

HIFU is an alternative to subfascial endoscopic perforating vein surgery (SEPS). HIFU can remove the need to move the focal spot along a long length of the vein, resulting in a procedure less complicated than SEPS. HIFU can be used to spot shrink the vein collagen just below the or near the fascia of the skin, providing the advantages of the deep perforator vein cuts applied in a SEPS procedure.

In some embodiments, HIFU is transmitted at least 4-12 cm below the surface of the skin. The HIFU probe targets superficial veins located just under the skin. Alternatively, HIFU is applied to treat other veins as well as hollow anatomical structures in general. Additionally, a HIFU probe has the capability of targeting the deep fascia below the surface of the skin and focusing its energy on a targeted treatment region of tissue. HIFU is powerful enough to treat deep veins located under the muscles. HIFU can also be used to treat perforating veins, blood vessels in the legs that connect the superficial leg veins to the deep leg veins.

In certain embodiments, ultrasound is applied using an ultrasound emitter. The ultrasound emitter is configured to emit ultrasound at multiple therapeutic ultrasound frequencies during a treatment cycle. The ultrasound emitter is configured to emit ultrasound at multiple levels of power: low-power ultrasound is applied during a low-power application phase, and high-power ultrasound is applied during a high-power application phase. In certain embodiments, HIFU is applied using an ultrasound probe. The external ultrasound probe comprises a therapy transducer movably mounted on a probe body. The external ultrasound probe is a handheld probe coupled to or including a power source for transmitting energy through the body surface and into the target blood vessel. The probe is applied to the skin or nearly to the skin, transmitting ultrasonic waves through the skin to the cells. The probe can be of any suitable size or shape known to one of skill in the art.

In certain embodiments, ultrasound is also applied using an acoustic coupler. The acoustic coupler is in sonic communication with the ultrasound emitter. The acoustic coupler has an acoustic coupling surface configured to contact a patient and facilitate delivery of ultrasound to the patient. The acoustic coupler provides a conduction path for ultrasound from the ultrasound emitter to the acoustic coupling surface. The acoustic coupler contains a displaceable acoustic coupling material. The acoustic coupler is configured to vary the length of the conduction path in accordance with variation in the thickness of the acoustic coupling material disposed between the emitter and the acoustic coupling surface.

In certain embodiments, the acoustic coupling material is flowable. Therefore, it will be appreciated that the presently disclosed technology includes an acoustic coupler comprising a flow path configured to allow the acoustic coupling material to flow in or out of the acoustic coupler, to facilitate varying the length of the conduction path. In some embodiments, the flow path extends from the acoustic coupler to a coupling material reservoir. The acoustic coupler comprises a compartment containing a fluid or mixture of fluid. The acoustic coupler comprises a gel. The acoustic coupling material comprises a degassed liquid. In certain embodiments, the acoustic coupler is a lens. In another embodiment, the acoustic coupler is a standoff.

In some embodiments, the ultrasound probe is used with an acoustic coupler or standoff. The acoustic coupler is positioned between the ultrasound probe and the skin. The acoustic coupler propagates the HIFU beam so as to place the beam at an optimal depth based on the location of the vein and the focal characteristics of the transducer. In some embodiments, the acoustic coupler alters the focusing beam characteristics, thus acting as a lens. In some embodiments, the acoustic coupler utilizes a reservoir system, which adjusts the standoff distance based on probe pressure to optimize the transducer-treatment zone distance. In other embodiments, the standoff consists of a gel or a compartment containing a fluid or mixture of fluids.

The HIFU probe is applied to the skin and directed toward a vein. In certain embodiments, the HIFU probe emits a focused high energy ultrasonic beam that generates heat. In some embodiments, the generation of heat by the ultrasonic beam causes necrosis of a portion of the vein and interrupts the blood flow through the vein. The HIFU probe uses the transmitted energy to heat the walls of the vein adequate to achieve the stable closure of the vein walls, thereby occluding the blood vessel. The generated heat causes tissue necrosis, vein wall collagen contraction, vein diameter reduction, and fibrotic occlusion.

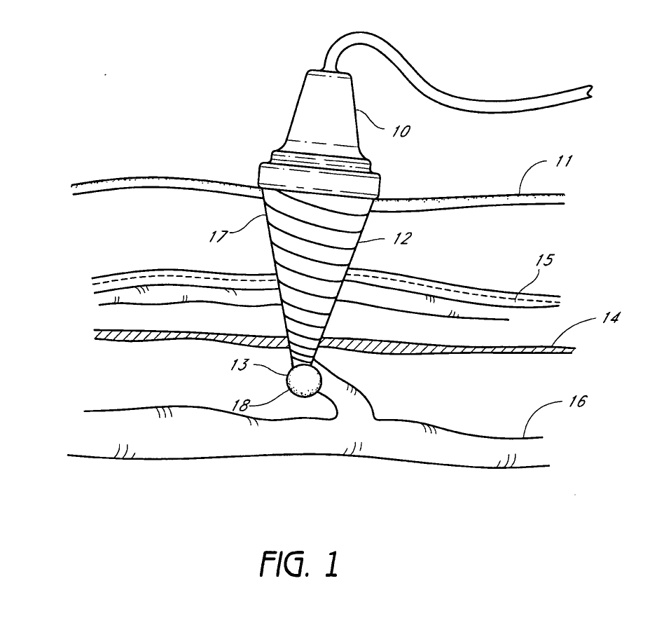

FIG. 1 shows an ultrasound probe 10 as it can be used to treat body tissue in accordance with certain embodiments of the methods and apparatus disclosed herein. The probe 10 is applied to the surface of the skin 11. The probe 10 emits high intensity focused ultrasound waves 17 through the surface of the skin 11 and into the underlying tissue. By penetrating the skin 11 at a depth between 4-12 cm under the epidermis, the HIFU wave 17 can reach and pass the depth of the superficial vein 15. The ultrasound probe 10 emits a wave 17 at a frequency able to penetrate the skin 11, the superficial vein 15, the deep fascia 14 under the skin 11, and a deep vein 16. The ultrasound wave can be emitted in a cone shape 12 that converges on a focal spot 13 underneath or near the deep fascia 14 of the skin 11. The focal spot 13 depicted in FIG. 1 is located just below the deep fascia 14 of the skin 11 and has a 1 mm-1 cm radius. The focal spot 13 preferably ranges in temperature between 60° C. and 120° C. A cavitation bubble 18 can be formed at the focal spot 13 and be employed to prevent propagation of heat past (deeper than) the focal spot 13.

In some embodiments, an HIFU apparatus provides the ability to guide or focus the treatment zone under ultrasound image guidance. Thus, the ability to image real time or semi-real time, the ability to dynamically change the focus depth (hot spot), and the ability to control the on-off delivery of the therapeutic levels of ultrasound is desirable. This apparatus is fabricated with known materials including phased array transducers, linear arrays, annular arrays, matrix arrays, or ring arrays and tools by those skilled in the art.

In some embodiments, a suitable coupling agent, such as an aqueous gel or degassed liquid, is applied between the ultrasound probe and the skin. FIG. 2 shows an acoustic coupler 21 applied between the HIFU transducer 23 and the skin 22. The acoustic coupler 21 is a gel that is applied to the surface of the skin 22. The volume of the gel 21 is selectively increased or decreased so as to set the optimal distance for the HIFU transducer 23. The HIFU transducer 23 is focused onto a treatment spot 24 that is located below the surface of the skin 22. Implementation of the gel 21 focuses the beam 25 emitted by the HIFU transducer onto a targeted treatment spot 24.

In one embodiment, the ultrasound-generating vein treatment apparatus functions in both a diagnostic and therapeutic mode. The system is first operated in the diagnostic imaging mode to locate the target vessel. Color Doppler can be employed to enhance location of the target vessel. The diagnostic probe is then switched out electronically, at which time the therapeutic mode is powered on. Sufficient acoustic power is then applied to cause heating and cellular disruption in the focal region of the transducer, thus sealing or reducing the blood flow through the vessel. In certain embodiments where ultrasound is applied to a perforator vein, application of ultrasound to the perforator vein disrupts the endothelium of the vein with the ultrasound. In some embodiments, the friction caused by the mechanical ultrasound wave provides a heating effect.

Design of the HIFU transducer can be customized with respect to frequency and focal depth so as to optimize the treatment region. In addition, specific algorithms for power application can be employed to increase efficacy while limiting unintended damage to adjacent tissue. In certain embodiments, the application of ultrasound is achieved by applying low-power ultrasound during a low-power application phase and high-power ultrasound during a high-power application phase.

One inherent difficulty in the use of HIFU is optimizing the distance between the transducer and treatment area so that the treatment area is within the focal region of the transducer. This allows the production of sufficient acoustic power density in the area to be treated. Methods to vary the focus electronically as with phased or annular transducers have been demonstrated. However, these designs suffer from complexity and high cost. In addition, with array geometries, undesired, spurious beam lobes cause undesired thermal effects outside of the perforator treatment area if they contain sufficient acoustic power. Array geometries are apodized to minimize grating and side lobes, however, the apodization affect the focusing of the main beam.

In some embodiments, a simplified method of maintaining proper focal distance is provided through the use of an ultrasound standoff. In certain embodiments, the ultrasound-generating vein treatment apparatus is equipped with a standoff that delivers the ultrasound. In certain embodiments, the ultrasound-generating vein treatment apparatus is equipped with a flow path in communication with the standoff to facilitate adjusting a standoff distance via acoustic coupling material flow to or from the standoff. In certain embodiments, the standoff is a hydrogel or a reservoir whereby the volume is selectively increased or decreased so as to set the optimal distance for the HIFU transducer.

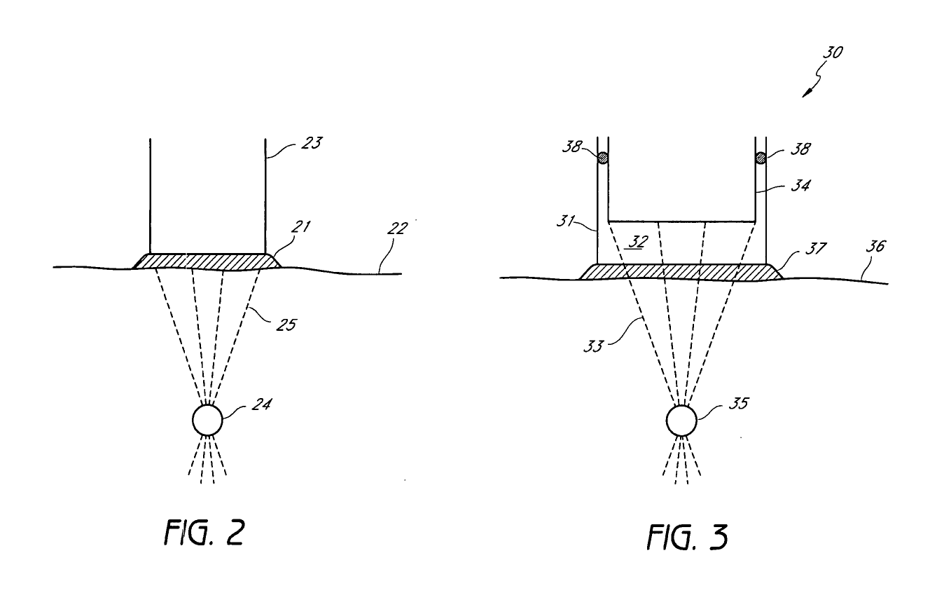

FIG. 3 shows an acoustic coupler comprising an ultrasound standoff 30 which permits adjustment of a standoff distance between the ultrasound transducer and the skin 36 or treatment area below the skin. The acoustic coupler comprises a reservoir system 31 filled partially with a liquid or gel 32, or any other suitable acoustic coupling material. The ultrasound (e.g. HIFU) transducer 34 is separated from the surface of the skin 36 by the reservoir system 31 filled with liquid or gel. As is evident from FIG. 3, a downward movement of the transducer 34 will displace the coupling material through flow paths to the sides of the beam path and into reservoirs located on the sides of the transducer 34.

Two slider seals 38 can be employed to separate the side walls of the transducer 34 from the side walls of the reservoir system 31. As the transducer 34 is moved downward and the coupling material enters the reservoirs, the seals 38 can slide upward to accommodate the entering material while preventing spillover. As the transducer 34 is moved upward, the acoustic coupling material flows back toward the beam path 33 and the seals 38 move downward correspondingly.

Variations on the arrangement of flow paths and reservoirs depicted in FIG. 3 are contemplated. For example, the number and type of reservoir(s) and flow path(s) can be varied. A reservoir can be provided in the form of a separate tank or bag which is in fluid communication with the standoff 30 via a flow path such as tubing or the like. Such a reservoir can comprise an elastic container which tends to urge any displaced acoustic coupling material contained in the reservoir back into the standoff upon upward movement of the transducer 34 within the standoff 30. The tubing or other flow path can connect to the standoff 30 on the side near the bottom, adjacent the beam path 33, or above the emitting surface of the transducer 34.

The liquid or gel 32 comprising the reservoir system 31 functions as an ultrasound transmission medium and focuses the beam 33 emitted by the transducer 34 onto a targeted treatment spot 35. Additionally, a layer of gel 37 can be applied on top of the surface of the skin 36 and separates the reservoir system 31 from the skin 36. Where employed, the layer of gel 37 further focuses the beam 33 emitted by the transducer 34 onto a targeted treatment spot 35.

The standoff 30 depicted in FIG. 3 advantageously facilitates a treatment system with a variable transducer standoff distance. Such a treatment system can advantageously be equipped with multiple transducer elements or otherwise configured to emit ultrasound at multiple frequencies. In operation, such a system can emit multiple frequencies simultaneously (including in a rapidly alternating fashion) to establish a pattern of multiple focal points or areas in the target tissue. When delivered at a treatment power level or other high power level, this pattern of foci can be moved up and down (or otherwise) by appropriate manipulation of the transducer 34 within the standoff 30, to ensure thorough treatment of the target area.

In another mode of operation, such a treatment system can first emit ultrasound at a first frequency at a diagnostic, imaging or other low power level. The resulting focal point or area can be moved up and down and observed by the user to determine whether the frequency and range of movement available are sufficient to place the focus in the desired treatment area. If it appears that the desired treatment area cannot be reached with the first frequency and the degree of movement afforded by the standoff 30, the transducer can be operated to emit ultrasound at a second frequency. As was done at the first frequency, the resulting second-frequency focal point or area can be moved up and down and observed by the user to determine whether the frequency and range of movement available are sufficient to place the second frequency focus in the desired treatment area. This can be repeated until a suitable treatment frequency or frequencies is determined. Upon determination of the suitable frequenc(ies), power can be delivered at a therapeutic power level at the selected frequenc(ies) to treat the desired vein(s) or other tissue. During therapeutic power delivery, the focal point or area (or pattern of foci) can be moved within the target area by appropriate manipulation of the transducer 34 within the standoff 31, as discussed above, to ensure treatment of the tissue.

A reservoir system, which adjusts the standoff distance based on probe pressure, can be employed to optimize the transducer-treatment zone distance. Since vasculature does not always reside in a plane equidistant from the skinline, the standoff can be designed in such a way so as to provide an angled wedge so that the therapy transducer is optimally focused along the treatment length. In other embodiments, depth adjustment is accomplished with a balloon connected directly (or not) to the HIFU probe to dynamically set the depth control by filling or un-filling with acoustic interface gel or fluid. Depth adjustment can also achieved by mechanical means, and control of the reservoir filling is accomplished manually or automatically.

Thermal coaptation, also known as tissue welding, of blood vessels is known to occur when vessel walls are held in contact during a process of controlled heating of the areas in contact. Simple manual compression of the perforator, inducing contact of the perforator walls, is effective to produce coaptation and thermal coagulation leading to vessel occlusion of the perforator while being heated in a controlled manner using HIFU. In certain embodiments, manual compression can be achieved near the perforator vein by using the ultrasound probe. In order to manually compress tissue, the location of a focal point of the ultrasound is adjusted. Exsanguination of veins can be accomplished using various techniques, including Trendelenburg positioning of the patient and external manual compression as well as techniques like Esmark bandage wrapping, VNUS echo-cuff, and tumescent anesthesia compression. The VNUS echo-cuff can be a useful apparatus for performing non-invasive transcutaneous HIFU over a wide field.

Subfascial or subcutaneous tumescence infiltration can be applied to provide protection from thermal injury to adjacent tissue and at the same time enhance the thermal coaptation of the perforator walls. In some embodiments, subfascial, perifascial or subcutaneous tumescent fluid is applied near the perforator veins. This tumescence serves to compress the perforator vein causing contact of the opposing walls and can be applied with or without manual compression. In certain embodiments where ultrasound is applied to a perforator vein, application of subfascial, perifascial or subcutaneous tumescent fluid near the perforator vein causes compression of the inner walls of the perforator vein toward each other with the tumescent fluid. The tumescent fluid causes compression of the inner walls of the vein toward each other. As HIFU is applied in the treatment region, and whereby said walls remain in intimate contact as heating occurs, the application of an appropriate ultrasound dosage results in durable coaptation of the vessel, effectively occluding it. The heat sinking effect of the surrounding tumescence can also serve to protect tissue not targeted for treatment.

Tumescent fluid can be applied to tissue near the vein in combination with the application of ultrasound to the vein, thereby occluding the vein with the ultrasound. Applying ultrasound to the vein propagates ultrasound through the tumescent fluid. Subfascial, perifascial or subcutaneous tumescent fluid is applied near the vein. In certain embodiments, occlusion of the vein occurs at a location below or near the deep fascia of the leg. In certain embodiments, the ultrasound employed in conjunction with tumescent fluid is high intensity focused ultrasound. In certain embodiments, applying ultrasound to the vein disrupts the endothelium of the vein with the ultrasound. In certain embodiments where ultrasound is applied to a perforator vein, the application of ultrasound to the perforator vein comprises propagating the ultrasound through the tumescent fluid. Tumescent fluid causes the surrounding tissue to become more homogenous to the ultrasound wave propagation.

Additionally, the applied tumescence can also serve to enhance the ultrasound beam path by providing better acoustic impedance matching from the skinline to the target perforator by displacing fat or fascial tissue. Infiltration of the tumescent around the target vessel also serves to make the subcutaneous tissue a more homogenous medium for the acoustical waves to propagate. Tumescence facilitates focusing the HIFU beam by establishing a more consistent refractive index. In some embodiments, it is desirable to have a slightly defocused beam to spread the area of the treatment zone, which makes clinical application of HIFU easier.

Although occlusion of the vessel is described above in terms of vessel wall coaptation and tissue welding, satisfactory occlusion of the vessel is also effected simply by the approximation of the vessel walls and the exsanguination of blood. In this approach, the HIFU thermal energy is used to ablate the treatment location and impart a durable fibrotic occlusion of the vessel by way of thermally induced collagen contraction and tissue coagulation rather than by the method of direct vessel wall coaptation and true tissue welding as described earlier. The vein is closed by shrinking the collagen.

In some embodiments, the focusing properties of the HIFU transducer determine the acoustic energy available in the focal region. For a circular aperture, the beamwidth is linearly proportional to the operating wavelength and focal point and inversely proportional to the effective diameter of the transducer aperture. Although these relationships determine the approximate beam characteristics in the focal region, other factors as well affect the focus of the transducer. In some embodiments, the invention includes a wide field transducer with a steep pressure angle to spread the focal spot to make the area of therapeutic heat more diffuse as well as change the shape of the focal spot for example from volumetrically round to volumetrically cylindrical or other.

The intervening coupling path, such as the standoff described earlier, consists of a gel or a compartment containing a primary fluid or mixture of fluids to alter the focusing beam characteristics in a predictable manner, thus acting as a lens. FIG. 3 shows an acoustic coupler comprising an ultrasound standoff. The ultrasound standoff can include a reservoir system 31 that is used between the transducer 34 and the skin 36 to adjust the focal depth. The fixed depth of focus is adjusted by applying downward pressure on the transducer 34 or upward pressure on the skin 36.

In some embodiments, the acoustic coupler is a lens. In some embodiments, the lens shape is flat. In some embodiments, the lens shape conforms to any curvature of the transducer. The fluid is optimized for properties such as density, propagation velocity, and attenuation so as to produce the optimal beam shape for the perforating vein treatment. In some embodiments both the curvature of the transducer and the standoff serve to focus the ultrasound beam. The transducer curvature provides the main focusing capabilities, and the standoff provides a fine tuning function. The standoff can be used to change other properties of the ultrasound beam, such as size and shape.

In addition to thermal effects known to occur under high power due to the high pressure fields present at the transducer focus, cavitation can also occur. In certain embodiments where ultrasound is applied to a perforator vein, the application of ultrasound to the perforator vein initiates cavitation in or near the perforator vein. In embodiments where tumescent fluid is applied to tissue near the vein and ultrasound is applied to the vein, applying the ultrasound initiates cavitation in or near the vein.

Cavitation is the growth and violent collapse of bubbles due to the acoustic pressure wave. Bubble effects disrupt precise energy deposition from the HIFU source. Excessive bubbles impede acoustic propagation, making it difficult to target the ultrasound leading to unpredictable beam shapes in the focal region of the transducer. Cavitation bubble formation prevents acoustic propagation of heat past the focal spot, targeted just below or near the fascia of the skin under ultrasound guidance and with minimal need to move the transducer head. Employing proper timing (adjusting the duration or the duty cycle) of the continuous wave excitation drives the HIFU transducer to produce predictable or reduced cavitation.

While cavitation is generally considered undesirable due to the reasons cited above, in some instances, intentionally induced cavitation produces desired physiological effects in the treated area. The collapse of the cavitation bubbles, should they be in contact with endothelial cells, causes lysing of these cells by violent disruption, augmenting the thermal effect of the high pressure HIFU wave to effect tissue coagulation and a durable fibrotic occlusion of the vessel.

Additionally, a standing cavitation bubble or coalescence of bubbles induced just beyond the focal spot of the acoustic waves in the diverging field can be used to limit the depth of beam penetration and thermal energy propagation. In certain embodiments where ultrasound is applied to a perforator vein, the application of ultrasound to the perforator vein limits the depth of ultrasound beam penetration by way of the cavitation that occurs in or near the perforator vein. In embodiments where tumescent fluid is applied to tissue near the vein and ultrasound is applied to the vein, cavitation limits the depth of ultrasound beam penetration. By placing the cavitation bubble(s) on the far side of the targeted tissue, the operator has the ability to better control the location of the thermal energy preventing heat energy from affecting non-target surrounding tissue. In some embodiments, the use of cavitation is employed in combination with tumescent compression effecting vessel exsanguination and/or manual external compression by pushing down or up on the ultrasound probe, which provides the ability to place the focal spot (therapeutic spot) on the far wall of the vessel and allows for the coapting of the vessel walls during treatment to effect an improved therapeutic outcome.

By creating a transducer design which emphasizes a very small focal region, excellent specificity is obtained for the treatment of very small perforating veins without the risk of thermal damage to adjacent tissue.

In some embodiments, the HIFU transducer employed to cause occlusion of the perforating vein is spherically focused, operating at a single frequency. Focusing is accomplished by creating a curvature to the transducer or by a focusing lens of suitable material applied to the front matching layer of the transducer or a combination of both.

Although a spherically focused transducer was previously described which ideally results in a single point focus or small circular area focus, in some embodiments, a transducer with a rectangular aperture is also employed. A transducer of this type ideally produces an ultrasound beam to be radiated in a line along the long axis of the transducer. In practice, a transducer of this type produces a beam with a rectangular cross-section. In some embodiments the transducer is curved along the short axis to improve focusing characteristics similar to a cylindrical lens. As described earlier, a suitable material applied to the transducer front matching layer acting as a lens, further enhances the beam characteristics.

In one method for treating an incompetent perforator, the transducer is spherically focused to a small circular region. While this type of focusing is implemented where greater specificity is desired, the use of the cylindrically focused transducer is employed where it is desired to treat a discrete segment of the vessel. Alternatively, where specificity is not as important, the transducer is defocused to create a larger treatment zone volume of the same inherent shape.

The HIFU transducer, by selection of piezoelectric material, the thickness of this material, and application of suitable impedance matching layers, allows for selecting the operating frequency to coincide with the series or parallel resonant frequency of a transducer for best electrical to acoustic conversion efficiency. The transducer is designed to work in conjunction with the variable standoff described above whereby some or all of the transducer properties are altered by the standoff design to optimize the acoustic beam used for treatment.

A further refinement of the transducer allows multiple resonances determined by the piezoelectric properties of the transducer and the matching layers. In this embodiment, two or more spaced resonant frequencies allow the transducer to efficiently convert electrical energy to acoustic energy and at the same time, allow the transducer to produce discrete focusing characteristics at the different resonances. In certain embodiments where ultrasound is applied to a perforator vein, the application of ultrasound to the perforator vein applies ultrasound at multiple frequencies. Additionally, in certain embodiments, ultrasound is applied at multiple levels of power: low-power ultrasound is applied at a low-power application phase, and high-power ultrasound is applied at a high-power application phase. In embodiments where tumescent fluid is applied to tissue near the vein and ultrasound is applied to the vein, the application of ultrasound applies ultrasound at multiple frequencies. The variation in focal depths among the frequencies allows treatment at different depths.

One method of treating a vein includes emitting multiple therapeutic ultrasound frequencies with an ultrasound probe. Multiple therapeutic ultrasound frequencies are applied to a treatment region that includes a portion of the vein. The distance between the ultrasound probe and the treatment region is adjusted while applying multiple therapeutic ultrasound frequencies. Applying multiple therapeutic ultrasound frequencies occludes the vein.

In certain embodiments, emitting multiple therapeutic ultrasound frequencies creates a pattern of multiple focal areas in the treatment region. Adjusting the distance between the ultrasound probe and the treatment region is achieved by moving the pattern of focal areas within the treatment region. In some embodiments, moving the pattern of focal areas within the treatment region entails moving the pattern along a direction of propagation of the ultrasound. In some embodiments, multiple therapeutic ultrasound frequencies are applied to a portion of a perforator vein. Additionally, in some embodiments, multiple therapeutic ultrasound frequencies are applied in conjunction with the application of subfascial, perifascial or subcutaneous tumescent fluid near the vein.

What is claimed is:

1. A method for treating a perforator vein, said method comprising:
applying ultrasound to said perforator vein; and
occluding said perforator vein with said ultrasound.

2. The method of claim 1, wherein said ultrasound is high intensity focused ultrasound (HIFU).

3. The method of claim 1, wherein occluding said perforator vein comprises occluding said vein at a location below or near the deep fascia of a leg.

4. The method of claim 1, wherein occluding said perforator vein comprises occluding said vein at a location below the deep fascia of a leg.

5. The method of claim 1, wherein applying ultrasound to said perforator vein comprises generating heat.

6. The method of claim 5, further comprising causing tissue necrosis, vein wall collagen contraction, vein diameter reduction and fibrotic occlusion, via said generated heat.

7. The method of claim 1, wherein applying ultrasound to said perforator vein comprises disrupting the endothelium of said vein with said ultrasound.

8. The method of claim 1, further comprising applying subfascial, perifascial or subcutaneous tumescent fluid near said perforator vein.

9. The method of claim 8, further comprising causing compression of the inner walls of said perforator vein toward each other with said tumescent fluid.

10. The method of claim 8, wherein applying ultrasound to said perforator vein comprises propagating said ultrasound through said tumescent fluid.

11. The method of claim 1, wherein applying ultrasound to said perforator vein comprises initiating cavitation in or near said perforator vein.

12. The method of claim 11, further comprising limiting the depth of ultrasound beam penetration with the cavitation.

13. The method of claim 1, wherein applying ultrasound comprises applying ultrasound at multiple frequencies.

14. The method of claim 1, wherein said ultrasound is applied via an ultrasound probe.

15. The method of claim 14, further comprising displacing said ultrasound probe along the direction of propagation of said ultrasound while applying said ultrasound.

16. The method of claim 14, further comprising manually compressing tissue near said perforator vein with said ultrasound probe.

17. The method of claim 16, wherein manually compressing comprises adjusting the location of a focal point of said ultrasound.

18. The method of claim 1, wherein applying ultrasound comprises applying low-power ultrasound during a low-power application phase and high-power ultrasound during a high-power application phase.

19. A method of facilitating perforator vein treatment with an ultrasound-generating vein treatment apparatus, said method comprising:
configuring said vein treatment apparatus to apply ultrasound to one or more perforator veins; and
configuring said vein treatment apparatus to occlude said one or more perforator veins with said ultrasound.

20. The method of claim 19, further comprising configuring said ultrasound-generating vein treatment apparatus to function in both a diagnostic and therapeutic mode.

21. The method of claim 19, further comprising equipping said ultrasound-generating vein treatment apparatus with a standoff to deliver said ultrasound.

22. The method of claim 21, further comprising equipping said ultrasound-generating vein treatment apparatus with a flow path in communication with said standoff to facilitate adjusting a standoff distance via material flow to or from said standoff.

23. A method of treating a vein, said method comprising:
applying tumescent fluid to tissue near said vein;
applying ultrasound to said vein; and
occluding said vein with said ultrasound.

24. The method of claim 23, wherein said ultrasound is high intensity focused ultrasound.

25. The method of claim 23, wherein occluding said vein comprises occluding said vein at a location below or near the deep fascia of a leg.

26. The method of claim 23, wherein occluding said vein comprises occluding said vein at a location below the deep fascia of a leg.

27. The method of claim 23, wherein applying ultrasound to said vein comprises disrupting the endothelium of said vein with said ultrasound.

28. The method of claim 23, wherein applying ultrasound to said vein comprises generating heat.

29. The method of claim 28, further comprising causing tissue necrosis, vein wall collagen contraction, vein diameter reduction and fibrotic occlusion, via said generated heat.

30. The method of claim 23, wherein applying said tumescent fluid further comprises applying subfascial, perifascial or subcutaneous tumescent fluid near said vein.

31. The method of claim 23, further comprising causing compression of the inner walls of said vein toward each other with said tumescent fluid.

32. The method of claim 23, wherein applying ultrasound to said vein comprises propagating said ultrasound through said tumescent fluid.

33. The method of claim 23, wherein applying ultrasound to said vein comprises initiating cavitation in or near said vein.

34. The method of claim 33, further comprising limiting the depth of ultrasound beam penetration with the cavitation.

35. The method of claim 23, wherein applying ultrasound comprises applying ultrasound at multiple frequencies.

36. The method of claim 23, wherein said vein is a perforator vein.

37. The method of claim 23, wherein applying ultrasound comprises applying low-power ultrasound during a low-power application phase and high-power ultrasound during a high-power application phase.

38. An apparatus for treating blood vessels, said apparatus comprising:
an ultrasound emitter, said ultrasound emitter being configured to emit ultrasound at multiple therapeutic ultrasound frequencies during a treatment cycle; and
an acoustic coupler in sonic communication with said emitter, said acoustic coupler having an acoustic coupling surface configured to contact a patient and facilitate delivery of ultrasound to said patient, said acoustic coupler providing a conduction path for ultrasound from said emitter to said acoustic coupling surface;
said acoustic coupler containing a displaceable acoustic coupling material;
said acoustic coupler being configured to vary the length of said conduction path in accordance with variation in the thickness of the acoustic coupling material disposed between said emitter and said acoustic coupling surface.

39. The apparatus of claim 38, wherein said acoustic coupling material is flowable and said acoustic coupler comprises a flow path configured to allow said acoustic coupling material to flow in or out of said acoustic coupler, to facilitate varying the length of said conduction path.

40. The apparatus of claim 39, wherein said flow path extends from said acoustic coupler to a coupling material reservoir.

41. The apparatus of claim 38, wherein said acoustic coupler comprises a compartment containing a fluid or mixture of fluid.

42. The apparatus of claim 38, wherein said acoustic coupling material comprises a gel.

43. The apparatus of claim 38, wherein said acoustic coupling material comprises a degassed liquid.

44. The apparatus of claim 38, wherein said acoustic coupler is a lens.

45. The apparatus of claim 38, wherein said acoustic coupler comprises a standoff.

46. A method of treating a vein, said method comprising:
emitting multiple ultrasound frequencies with an ultrasound probe;
applying said multiple ultrasound frequencies to a treatment region that includes a portion of said vein;
adjusting the distance between said ultrasound probe and said treatment region while applying said multiple ultrasound frequencies; and
occluding said vein with said multiple ultrasound frequencies.

47. The method of claim 46, wherein emitting multiple ultrasound frequencies comprises creating a pattern of multiple focal areas in said treatment region; and
adjusting said distance comprises moving said pattern of focal areas within said treatment region.

48. The method of claim 47, wherein moving said pattern of focal areas comprises moving said pattern along a direction of propagation of said ultrasound.

49. The method of claim 46, wherein said vein is a perforator vein.

50. The method of claim 46, further comprising applying subfascial, perifascial or subcutaneous tumescent fluid near said vein.

51. A method comprising:
emitting a first ultrasound frequency with an ultrasound probe;
applying said first ultrasound frequency to a first target region near a vein;
adjusting the distance between said ultrasound probe and said first target region while applying said first ultrasound frequency;
after applying said first ultrasound frequency, emitting a second ultrasound frequency with said ultrasound probe;
applying said second ultrasound frequency to a second target region near said vein; and
adjusting the distance between said ultrasound probe and said treatment region while applying said second ultrasound frequency.

52. The method of claim 51, further comprising selecting one of said first frequency and said second frequency for further application of ultrasound to said vein at said selected frequency.

53. The method of claim 52, further comprising applying ultrasound at said selected frequency to said vein at a power level sufficient to occlude said vein.

54. The method of claim 52, further comprising observing a focal point or area of said applied first and second ultrasound frequencies; wherein said selecting comprises selecting based on said observing.

55. The method of claim 51, wherein said second target region at least overlaps said first target region.

56. The method of claim 51, wherein adjusting the distance between said ultrasound probe and said first target region comprises moving a focal point or area of said applied first ultrasound relative to said first target region.

57. A method comprising:
emitting low-power ultrasound with an ultrasound probe;
applying said low-power ultrasound to a target region near a vein;
adjusting the distance between said ultrasound probe and said target region while applying said low-power ultrasound;
after applying said low-power ultrasound, applying high-power ultrasound to said target region to occlude said vein.

58. The method of claim 57, wherein adjusting the distance comprises placing a focal point or region of said low-power ultrasound at a desired treatment location.

59. The method of claim 58, further comprising applying said high-power ultrasound to said desired treatment location.

60. The method of claim 57, further comprising observing a focal point or region of said low-power ultrasound, and aiming said high-power ultrasound with based on the location of said focal point or region.

61. The method of claim 57, wherein said vein comprises a perforator vein.
